(19) United States
(12) Patent Application Publication
(10) Pub. No.: US 2021/0023099 A1
(43) Pub. Date: Jan. 28, 2021

(54) COMBINATION THERAPY WITH A BET INHIBITOR AND A PROTEASOME INHIBITOR

(71) Applicant: Hoffmann-La Roche Inc., Little Falls, NJ (US)

(72) Inventors: Mark D. Demario, New York, NY (US); Thomas Friess, Penzberg (DE); Astrid Alexandra Ruefli-Brasse, San Mateo, CA (US)

(73) Assignee: Hoffmann-La Roche Inc., Little Falls, NJ (US)

(21) Appl. No.: 17/071,953

(22) Filed: Oct. 15, 2020

Related U.S. Application Data
(63) Continuation of application No. PCT/EP2019/059719, filed on Apr. 16, 2019.
(60) Provisional application No. 62/659,207, filed on Apr. 18, 2018.

(51) Int. Cl. A61K 31/69; A61K 31/551; A61P 35/00

(52) U.S. Cl. CPC A61K 31/69; A61P 35/00; A61K 31/551

(57) ABSTRACT

The present invention is directed to the combination therapy, in particular of multiple myeloma, with a BET inhibitor and a proteasome inhibitor.

This application is a continuation of International Application No. PCT/EP2019/059719 having an International filing date of Apr. 16, 2019, which claims benefit of and priority to U.S. Provisional Application No. 62/659,207, filed Apr. 18, 2018, all of which are incorporated by reference in their entirety.

The present invention is directed to the combination therapy, in particular of multiple myeloma, with a BET inhibitor and a proteasome inhibitor.

Multiple myeloma (MM) is a debilitating malignancy that is part of a spectrum of diseases ranging from monoclonal gammopathy of unknown significance (MGUS) to plasma cell leukemia. First described in 1848, MM is characterized by a proliferation of malignant plasma cells and a subsequent overabundance of monoclonal paraprotein (M protein).

The presentation of MM can range from asymptomatic to severely symptomatic, with complications requiring emergent treatment. Systemic ailments include bleeding, infection, and renal failure; pathologic fractures and spinal cord compression may occur.

Epigenetic dysregulation plays an important role in driving the aberrant gene expression patterns seen in a variety of hematologic malignancies. As many epigenetic alterations are reversible, these factors have drawn considerable attention as potential antineoplastic targets. One particular target of significant clinical interest is the bromodomain and extra-terminal (BET) family of proteins, which includes BRD2, BRD3, BRD4, and the testis-specific BRDT. Bromodomains (BRDs) are protein domains that possess a high affinity for binding to acetylation motifs, including acetylated histone proteins within chromatin. The BET family of proteins binds to acetylated chromatin and regulates gene transcription.

Selective inhibition of the interaction between BET proteins and acetylated chromatin has resulted in significant activity in preclinical models of acute leukemia, lymphoma, and multiple myeloma (MM). Targeting BET proteins could specifically target transcription of oncogenes and genes critical to disease development and progression.

Degradation of cellular proteins is a tightly regulated and complex process that plays a central role in regulating cellular function and maintaining homoeostasis. The ubiquitin-proteasome pathway (UPP) represents the major pathway for intracellular protein degradation. The majority of proteins are degraded through this pathway, including those involved in the regulation of numerous cellular and physiological functions, such as cell cycle, apoptosis, transcription, DNA repair, protein quality control and antigens.

Defects within the UPP pathway are associated with a number of diseases, including cancer; thus inhibitors of this pathway should prevent malignant cells from proliferation. Proteasome inhibitors are drugs that block the proper action of proteasomes namely degradation of pro-apoptotic factors such as the p53 protein, permitting activation of programmed cell death in neoplastic cells dependent upon suppression of pro-apoptotic pathways.

It was surprisingly found that the combination of a BET inhibitor with a proteasome inhibitor showed significantly enhanced efficacy against multiple myeloma, causing a distinct tumor regression. Surprisingly, the tumor regression with this combination is more than additive, i.e. superior to the cumulated anti-tumor efficacy induced by each of the two components separately.

The invention thus relates in particular to:

A BET inhibitor and a proteasome inhibitor for use as a medicament;

A BET inhibitor and a proteasome inhibitor for use in the treatment of multiple myeloma;

The BET inhibitor and proteasome inhibitor for use according to the invention, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146), INCB-054329, INCB-057643, GSK525762, GS-5829, CPI-0610, Birabresib, PLX51107, ABBV-075, BI 894999, FT-1101, ZEN-3694, GSK-2820151 or BMS-986158;

The BET inhibitor and proteasome inhibitor for use according to the invention, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146);

The BET inhibitor and proteasome inhibitor for use according to the invention, wherein the proteasome inhibitor is bortezomib, carfilzomib, ixazomib, oprozomib, delanzomib or marizomib.

The BET inhibitor and proteasome inhibitor for use according to the invention, wherein the proteasome inhibitor is bortezomib;

The BET inhibitor and proteasome inhibitor for use according to the invention wherein the BET inhibitor is for subcutaneous administration;

The BET inhibitor and proteasome inhibitor for use according to the invention wherein the proteasome inhibitor is for intravenous administration;

The BET inhibitor and proteasome inhibitor for use according to the invention, comprising one or more additional other cytotoxic, chemotherapeutic or anti-cancer agents;

The BET inhibitor and proteasome inhibitor for use according to the invention, comprising ionizing radiation enhancing the effects of said agents;

A pharmaceutical composition comprising a BET inhibitor and a proteasome inhibitor and one or more pharmaceutically acceptable excipients;

A pharmaceutical composition comprising a BET inhibitor and a proteasome inhibitor and one or more pharmaceutically acceptable salt thereof for use in the treatment of multiple myeloma;

The use of a BET inhibitor and a proteasome inhibitor for the manufacture of a medicament for the treatment of multiple myeloma;

The use of a BET inhibitor and a proteasome inhibitor in the treatment of multiple myeloma;

A method of treatment of multiple myeloma comprising the administration of a BET inhibitor and a proteasome inhibitor to a patient in the need thereof;

A kit comprising a BET inhibitor and a proteasome inhibitor for the simultaneous, separate or sequential administration of said BET inhibitor and proteasome inhibitor;

A kit according to the invention wherein the BET inhibitor is for subcutaneous administration and the proteasome inhibitor is for intravenous administration;

A kit according to the invention for use in the treatment of multiple myeloma;

A pharmaceutical composition, a use, a method or a kit according to the invention, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146), INCB-054329, INCB-057643, GSK525762, GS-5829, CPI-0610, Birabresib, PLX51107, ABBV-075, BI 894999, FT-1101, ZEN-3694, GSK-2820151 or BMS-986158;

A pharmaceutical composition, a use, a method or a kit according to the invention, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146);

A pharmaceutical composition, a use, a method or a kit according to the invention, wherein the proteasome inhibitor is bortezomib, carfilzomib, ixazomib, oprozomib, delanzomib or marizomib; and

A pharmaceutical composition, a use, a method or a kit according to the invention, wherein the proteasome inhibitor is bortezomib.

The BET inhibitor and proteasome inhibitor according to the invention are thus administered in combination.

The invention thus relates to a BET inhibitor and a proteasome inhibitor for use in combination according to the invention.

The invention thus relates to a BET inhibitor and a proteasome inhibitor for use in combination as a medicament, in particular for use in combination in the treatment of multiple myeloma.

In one embodiment, the BET inhibitor is a compound selected from the compounds described in WO 2011/143669. Methods of producing said BET inhibitors are also disclosed in WO 2011/143669.

Most preferably, the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide as in the formula below, or a salt thereof. Example JQ35 of WO 2011/143669 describes a method for its preparation.

The preferred BET inhibitor is depicted in the following formula:

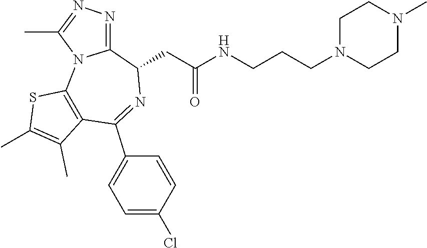

The above BET inhibitor is also known as RG6146, JQ35 or TEN-010.

In one embodiment, the proteasome inhibitor is a compound selected from the compounds described in U.S. Pat. No. 6,713,446 B2 or U.S. Pat. No. 6,958,319 B2. Methods of producing said proteasome inhibitors are also disclosed in U.S. Pat. No. 6,713,446 B2 or 6958319 B2.

Most preferably, the proteasome inhibitor is [(1R)-3-methyl-1-[[(2S)-3-phenyl-2-(pyrazine-2-carbonylamino)propanoyl]amino]butyl]boronic acid, also named N-(2-pyrazine)carbonyl-L-phenylalanine-L-leucine boronic acid, as in the formula below. It is formulated as a D-mannitol boronic ester. U.S. Pat. No. 6,713,446 B2 describes methods for preparation of said proteasome inhibitor.

The preferred proteasome inhibitor is depicted in the following formula:

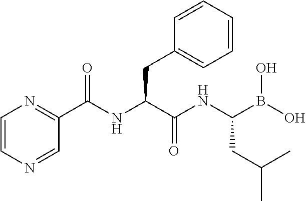 [SMILES: CC(C)C[C@H](NC(=O)[C@H](Cc1ccccc1)NC(=O)c1cnccn1)B(O)O]

The above proteasome inhibitor is also named bortezomib or PS-341 and is available under the tradenames Velcade, Neomib and Bortecad.

### BRIEF DESCRIPTION OF THE FIGURES

FIG. 1: Antitumor efficacy of therapy (MM1S) with the double combination of RG6146 and bortezomib compared to vehicule and mono therapies (Days 14-28).

FIG. 2: Antitumor efficacy of therapy (OPM-2) with the double combination of RG6146 and bortezomib compared to vehicule and mono therapies (Days 17-31).

The term “BET inhibitor” according to the invention refers to agents that prevent activity of BET proteins with an IC₅₀ of about 0.001 μM to about 2 μM.

The term “proteasome inhibitor according to the invention refers to agents that prevents activity of proteasomes with an IC₅₀ of about 0.001 μM to about 2 μM.

“Salt” refers to salts of the compounds as a pharmaceutically acceptable salt. Such salts can be exemplified by the salts with alkali metals (potassium, sodium, and the like), salts with alkaline-earth metals (calcium, magnesium, and the like), the ammonium salt, salts with pharmaceutically acceptable organic amines (tetramethylammonium, triethylamine, methylamine, dimethylamine, cyclopentylamine, benzylamine, phenethylamine, piperidine, monoethanolamine, diethanolamine, tris(hydroxymethyl)aminomethane, lysine, arginine, N-methyl-D-glucamine, and the like), and acid addition salts (inorganic acid salts (the hydrochloride, hydrobromide, hydroiodide, sulfate, phosphate, nitrate, and the like) and organic acid salts (the acetate, trifluoroacetate, lactate, tartrate, oxalate, fumarate, maleate, benzoate, citrate, methanesulfonate, ethanesulfonate, benzenesulfonate, toluenesulfonate, isethionate, glucuronate, gluconate, and the like)).

“IC₅₀” refers to the concentration of a particular compound required to inhibit 50% of a specific measured activity.

The terms “combination”, “co-administration” or “co-administering” refer to the administration of the BET inhibitor and the proteasome inhibitor according to the invention in one or several formulations. The co-administration can be simultaneous or sequential in either order, wherein preferably there is a time period while both (or all) active agents simultaneously exert their biological activities. The BET inhibitor and the proteasome inhibitor can be co-administered either simultaneously or sequentially. When the therapeutic agents are co-administered sequentially, the dose can for example be administered either on the same day in three separate administrations, or one of the agents can be administered on day 1 and the second and third can be co-administered on day 2 to day 7, preferably on day 2 to 4. Thus in one embodiment the term “sequentially” means within 7 days after the dose of the first component, preferably within 4 days after the dose of the first component; and the term “simultaneously” means at the same time or on the same day. The terms “co-administration” with respect to the maintenance doses of the BET inhibitor and the proteasome inhibitor mean that the maintenance doses can be either co-administered simultaneously, if the treatment cycle is appropriate for both drugs, e.g. every week. Or one of the components (either the proteasome inhibitor or the BET inhibitor) can be administered e.g. every first to third day and the second component can be administered every week. Or the maintenance doses are co-administered sequentially, either within one or within several days.

It is self-evident that the inhibitors are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

The amount of co-administration of the BET inhibitor and the proteasome inhibitor and the timing of co-administration will depend on the type (species, gender, age, weight, etc.) and condition of the patient being treated and the severity of the disease or condition being treated.

The BET inhibitor is preferably administered subcutaneously.

The daily doses of the BET inhibitor indicated below are daily doses on days of dosing.

The BET inhibitor is preferably administered at a dose between about 0.3 mg/kg/d and about 0.65 mg/kg/d.

The BET inhibitor is preferably administered daily for 14 consecutive days every 3 weeks (i.e. 2 weeks of dosing, 1 week of rest).

The BET inhibitor is preferably administered subcutaneously, at a dose between about 0.3 mg/kg/d and about 0.65 mg/kg/d.

The BET inhibitor is preferably administered subcutaneously, at a dose between about 0.3 mg/kg/d and about 0.65 mg/kg/d for 14 consecutive days every 3 weeks (i.e. 2 weeks of dosing, 1 week of rest).

The BET inhibitor is preferably RG6146.

The administration of the BET inhibitor, in particular RG6146, can be interrupted for up to 3 weeks, i.e. 1, 2 or 3 weeks.

The proteasome inhibitor is preferably administered by subcutaneous or intravenous injection (i.v.).

The daily doses of the proteasome inhibitor indicated below are daily doses on days of dosing.

The proteasome inhibitor is preferably administered at a dose between about 0.7 mg/m² and 1.3 mg/m² (body surface area) per day on days of dosing.

The dose of 1.3 mg/m² is preferred. However, lower doses, like e.g. about 0.7 mg/m² or about 1.0 mg/m² can be used if the dose of 1.3 mg/m² is not tolerated over time, for example in case of cumulative toxicities.

The proteasome inhibitor is preferably administered i.v., at a dose between about 0.7 mg/m2 and 1.3 mg/m² per day on days of dosing.

The proteasome inhibitor can advantageously be administered twice weekly for two weeks, advantageously on days 1, 4, 8, and 11 in a 21-day treatment cycle. This 3-week period is considered a treatment cycle. It is recommended that patients receive 2 cycles of the proteasome inhibitor following a confirmation of a complete response. It is also recommended that responding patients who do not achieve a complete remission receive a total of 8 cycles of proteasome inhibitor therapy.

At least 72 hours should preferably elapse between consecutive doses of the proteasome inhibitor.

The proteasome inhibitor is preferably bortezomib.

The administration cycles of the BET inhibitor and proteasome inhibitor are preferably initiated on the same day.

Depending on the type and severity of the disease, the following amounts can be administered: about 0.3 mg/kg/d to about 0.65 mg/kg/d of the BET inhibitor on days of dosing, preferably RG6146; about 0.7 mg/m²/d to about 1.3 mg/m²/d (body surface area) on days of dosing, preferably bortezomib.

A particular advantageous combination is about 0.3 mg/kg/d to about 0.65 mg/kg/d of the BET inhibitor, preferably RG6146, every day for 14 consecutive days every 3 weeks (i.e. 2 weeks of dosing, 1 week of rest); about 0.7 mg/m²/d to about 1.3 mg/m²/d of the proteasome inhibitor, preferably bortezomib, twice weekly for two weeks every 3 weeks.

A further particular advantageous combination is about 0.3 mg/kg/d to about 0.65 mg/kg/d of the BET inhibitor, preferably RG6146, subcutaneously every day for 14 consecutive days every 3 weeks (i.e. 2 weeks of dosing, 1 week of rest); about 0.7 mg/m²/d to about 1.3 mg/m²/d of the proteasome inhibitor, preferably bortezomib, subcutaneously or intravenously twice weekly for two weeks every 3 weeks (i.e. 2 weeks of dosing, 1 week of rest).

In the above dosing regimens, the administration of the BET inhibitor, in particular RG6146, can be interrupted for up to 3 weeks, i.e. 1, 2 or 3 weeks.

The recommended doses may vary when there is a further co-administration of a chemotherapeutic agent.

The present invention is useful for preventing or reducing metastasis or further dissemination in such a patient suffering from multiple myeloma. This invention is useful for increasing the duration of survival of such a patient, increasing the progression free survival of such a patient, increasing the duration of response, resulting in a statistically significant and clinically meaningful improvement of the treated patient as measured by the duration of survival, progression free survival, response rate or duration of response. In a preferred embodiment, this invention is useful for increasing the response rate in a group of patients.

In the context of this invention, additional other cytotoxic, chemotherapeutic or anti-cancer agents, or compounds or ionizing radiation that enhance the effects of such agents (e.g. cytokines) may be used. Such molecules are suitably present in combination in amounts that are effective for the purpose intended.

Such additional agents include, for example: alkylating agents or agents with an alkylating action, such as cyclophosphamide (CTX; e.g. Cytoxan®), chlorambucil (CHL; e.g. Leukeran®), cisplatin (CisP; e.g. Platinol®) busulfan (e.g. Myleran®), melphalan, carmustine (BCNU), streptozotocin, triethylenemelamine (TEM), mitomycin C, and the like; anti-metabolites, such as methotrexate (MTX), etoposide (VP16; e.g. Vepesid®), 6-mercaptopurine (6MP), 6-thiocguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. Adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. Taxol®) and paclitaxel derivatives, the cytostatic agents, glucocorticoids such as dexamethasone (DEX; e.g. Decadron®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. Ethyol®), dactinomycin, mechlorethamine (nitrogen mustard), streptozocin, cyclophosphamide, lomustine (CCNU), doxorubicin lipo (e.g. Doxil®), gemcitabine (e.g. Gemzar®), daunorubicin lipo (e.g. Daunoxome®), procarbazine, mitomycin, docetaxel (e.g. Taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine or chlorambucil.

The use of the cytotoxic and anticancer agents described above as well as antiproliferative target-specific anticancer drugs like protein kinase inhibitors in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

In the context of this invention, an effective amount of ionizing radiation may be carried out and/or a radiopharmaceutical may be used. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, yttrium-90, cesium-137, iridium-192, americium-241, gold-198, cobalt-57, copper-67, technetium-99, iodine-123, iodine-131, and indium-111.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week.

In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in WO 99/60023.

As used herein, a “pharmaceutically acceptable carrier” or “pharmaceutically acceptable excipient” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Pharmaceutical compositions can be obtained by processing the BET inhibitor and the proteasome inhibitor according to this invention with pharmaceutically acceptable, inorganic or organic carriers or excipients. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions of the BET inhibitor and the proteasome inhibitor, alone or in combination, can be prepared for storage by mixing the active ingredient having the desired degree of purity with optional pharmaceutically acceptable carriers, excipients or stabilizers (Remington's Pharmaceutical Sciences 16th edition, Osol, A. (ed.) (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN′, PLURONICS™ or polyethylene glycol (PEG).

Pharmaceutical compositions of the BET inhibitor and of the proteasome inhibitor include those suitable for oral, nasal, topical (including buccal and sublingual), rectal, vaginal and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a proteasome inhibitor or a BET inhibitor which produces a therapeutic effect. Generally, out of one hundred percent, this amount will range from about 1 percent to about 90 percent of active ingredient, preferably from about 5 percent to about 70 percent, most preferably from about 10 percent to about 30 percent. Methods of preparing these compositions include the step of bringing into association a proteasome inhibitor or a BET inhibitor with the carrier and, optionally, one or more accessory ingredients. In general, the pharmaceutical compositions can be prepared by uniformly and intimately bringing into association a proteasome inhibitor and a BET inhibitor with liquid carriers, or finely divided solid carriers, or both, and then, if necessary, shaping the product. Pharmaceutical compositions suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a proteasome inhibitor and a BET inhibitor as an active ingredient. A proteasome inhibitor and a BET inhibitor may also be administered as a bolus, electuary or paste.

In further embodiments of the invention, the BET inhibitor and the proteasome inhibitor are formulated into one or two separate pharmaceutical compositions.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. (ed.) (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT′ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

The following examples and figures are provided to illustrate the invention and have no limiting character.

### EXAMPLES

### Example 1: In Vivo Antitumor Efficacy (MM1s)

The in vivo antitumor efficacy of BET inhibitor RG6146 in combination with proteasome inhibitor bortezomib was evaluated against MM1S MM xenografts.

Test Agents

BET inhibitor RG6146 was provided as a powder from Roche, Basel, Switzerland and resuspended prior to use. Proteasome inhibitor bortezomib was provided by MedChem Express, NJ, USA and formulated prior to use.

Cell Line and Culture Conditions

The original MM1s human Multiple Myeloma cell line (MM) was purchased from ATCC (Manassas, Va., USA). Expansion of tumor cells for the transplantation was done by the TAP CompacT CellBase Cell Culture Roboter according to the protocol. Tumor cell line was routinely cultured in RPMI 1640 medium, FCS 10% and L-Glutamin 2 mM at 37° C. in a water-saturated atmosphere at 5% CO2. Culture passage was performed with trypsin/EDTA 1× splitting twice/week and passage 2 used for transplantation.

Animals

Female CIEA NOG mice (Taconic), age 5-6 weeks at arrival, maintained under specific-pathogen-free condition with daily cycles of 12 h light/12 h darkness according to committed guidelines. Experimental study protocol was reviewed and approved by local government. After arrival animals were maintained in animal facility for one week to get accustomed to new environment and for observation. Continuous health monitoring was carried out on regular basis. Diet food and autoclaved water were provided ad libitum.

Monitoring

Animals were controlled daily for clinical symptoms and detection of adverse effects. For monitoring throughout the experiment body weight of animals was documented.

Treatment of Animals

Animal treatment started after randomisation when median tumor size was about 100 mm³. The vehicle was administered ip once daily (QD) on D14-28. BET inhibitor RG6146 ip treatment at 30 mg/kg was done as single agent and in combination on D14-28. Finally, proteasome inhibitor bortezomib was given i.v. at 0.5 mg/kg as single agent and in combination twice a week for two weeks.

Antitumor Efficacy

MM1s human MM cells were s.c. inoculated with Matrigel onto female CIEA-NOG mice. Tumor bearing mice were randomized 14 days later to the indicated study groups and compound treatment initiated. Tumor bearing animals were treated with vehicle control, with the BET inhibitor RG6146 at 30 mg/kg or with proteasome inhibitor bortezomib at 0.5 mg/kg as single agent and in combination thereof. As a result, RG6146 given as single agent demonstrated significant anti-tumor efficacy against MM1s xenografts while bortezomib was slightly active. Briefly, treatment with the BET inhibitor RG6146 resulted in strong significant efficacy with 83% tumor growth inhibition against MM1s xenografts compared to control. In contrast to this, a low activity was noticed after treatment with the proteasome inhibitor bortezomib (29% TGI), whereas superior efficacy was achieved after treatment with the dual combination group including the BET inhibitor RG6146 plus proteasome inhibitor bortezomib.

In more detail the dual combination approach substantially induced tumor regression which reached finally 50%. The strong efficacy of the dual combination arm with tumor regression of MM1s xenografts was more than additive compared to the respective single agent arms.

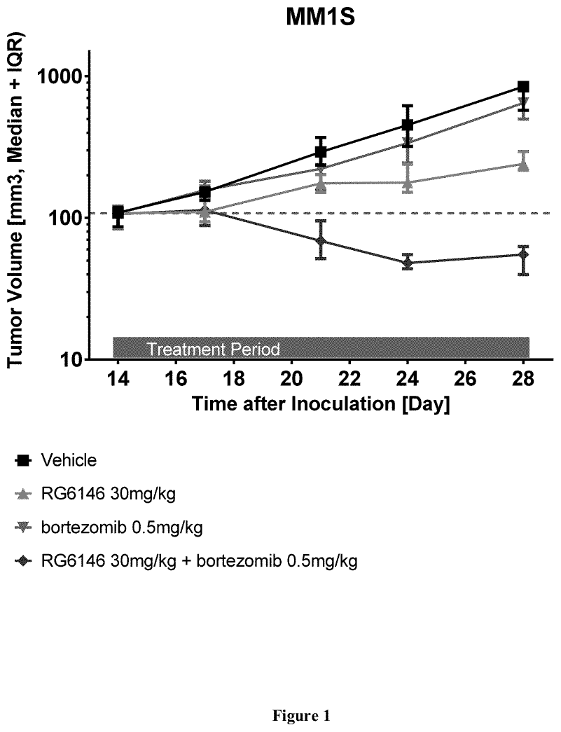

The results are illustrated by Table 1 below and FIG. 1.

TABLE 1
Efficacy of BETi RG6146 and proteasome inh. bortezomib (Day 28)

| | | | | Tumor | |
|---|---|---|---|---|---|
| | Dose | | TGI | Regression | npTCR |
| Compound | (mg/kg) | Schedule | % | % | and CI |
| control | — | ip | — | — | — |
| RG6146 | 30 | ip | 83 | 0 | 0.29 |
| | | (QD) | | | (CI 0.21-0.58) |
| bortezomib | 0.5 | iv | 29 | 0 | 0.75 |
| | | (2 × Q7D) | | | (CI 0.55-1.27) |
| RG6146 | 30 | ip | >100 | 50 | 0.07 |
| | | (QD) | | | |
| bortezomib | 0.5 | iv | | | (CI 0.05-0.12) |
| | | (2 × Q7D) | | | |

TCR: Treatment to Control Ratio;
pTCR: non-parametric Tumor Control Ratio;
CI: Confidence Interval;
QD: every day;
Q7D: every seven day
TGI: Tumor growth inhibition

### Example 2: In Vivo Antitumor Efficacy (OPM-2)

The in vivo antitumor efficacy of BET inhibitor RG6146 in combination with proteasome inhibitor bortezomib was evaluated against OPM-2 MM xenografts.

Test Agents

BET inhibitor RG6146 was provided as a powder from Roche, Basel, Switzerland and resuspended prior to use. Proteasome inhibitor bortezomib was provided by MedChem Express, NJ, USA and formulated prior to use.

Cell Line and Culture Conditions

The original OPM-2 human Multiple Myeloma cell line (MM) was purchased from ATCC (Manassas, Va., USA). Expansion of tumor cells for the transplantation was done by the TAP CompacT CellBase Cell Culture Roboter according to the protocol. Tumor cell line was routinely cultured in RPMI 1640 medium, FCS 10% and L-Glutamin 2 mM at 37° C. in a water-saturated atmosphere at 5% CO2. Culture passage was performed with trypsin/EDTA 1× splitting twice/week and passage 2 used for transplantation.

Animals

Female Scid beige mice (Charles River), age 6-7 weeks at arrival, maintained under specific-pathogen-free condition with daily cycles of 12 h light/12 h darkness according to committed guidelines. Experimental study protocol was reviewed and approved by local government. After arrival animals were maintained in animal facility for one week to get accustomed to new environment and for observation. Continuous health monitoring was carried out on regular basis. Diet food and autoclaved water were provided ad libitum.

Monitoring

Animals were controlled daily for clinical symptoms and detection of adverse effects. For monitoring throughout the experiment body weight of animals was documented.

Treatment of Animals

Animal treatment started after randomisation when median tumor size was about 115 mm³. The vehicle was administered ip once daily (QD) on D17-31. BET inhibitor RG6146 ip treatment at 30 mg/kg was done as single agent and in combination on D17-31. Finally, proteasome inhibitor bortezomib was given i.v. at 0.5 mg/kg as single agent and in combination twice a week for two weeks.

Antitumor Efficacy

OPM-2 human MM cells were s.c. inoculated with Matrigel onto female Scid beige mice. Tumor bearing mice were randomized 17 days later to the indicated study groups and compound treatment initiated. Tumor bearing animals were treated with vehicle control, with the BET inhibitor RG6146 at 30 mg/kg or with proteasome inhibitor bortezomib at 0.5 mg/kg as single agent and in combination thereof. As a result, RG6146 given as single agent demonstrated significant anti-tumor efficacy against OPM-2 xenografts while bortezomib was less active. Briefly, treatment with the BET inhibitor RG6146 resulted in strong significant efficacy with 85% tumor regression against OPM-2 xenografts compared to control and 5 out of 10 mice with complete tumor remission. In contrast to this, low activity was noticed after treatment with the proteasome inhibitor bortezomib (60% TGI), whereas superior efficacy was achieved after treatment with the dual combination group including the BET inhibitor RG6164 plus proteasome inhibitor bortezomib.

In more detail the dual combination approach substantially induced tumor regression and complete tumor remission in all mice (10/10 tumor free). The strong efficacy of the dual combination arm with tumor regression of OPM-2 xenografts was more than additive compared to the respective single agent arms.

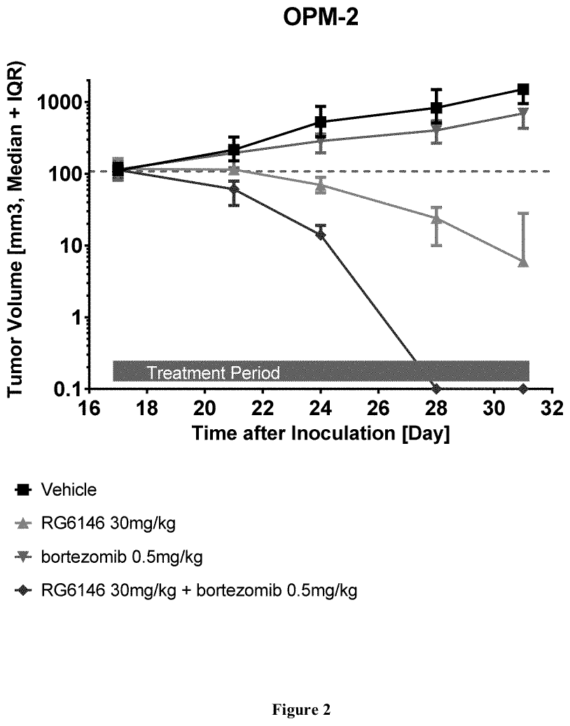

The results are illustrated by Table 2 below and FIG. 2.

TABLE 2
Efficacy of BETi RG6146 and proteasome inh. bortezomib (Day 31)

| | | | | Tumor | npTCR | Tumor |
|---|---|---|---|---|---|---|
| | Dose | | TGI | Regression | and | free |
| Compound | (mg/kg) | Schedule | % | % | CI | animal |
| control | — | ip | — | — | — | — |
| RG6146 | 30 | ip | >100 | 85 | 0.008 | 5/10 |
| | | (QD) | | | (CI 0.0- | |
| | | | | | 0.02) | |
| bortezomib | 0.5 | iv | 60 | 0 | 0.34 | 0/10 |
| | | (2xQ7D) | | | (CI 0.18- | |
| | | | | | 0.74) | |
| RG6146 | 30 | ip (QD) | >100 | 100 | 0.00 | 10/10 |
| bortezomib | 0.5 | iv (2xQ7D) | | | (CI 0.00- | |
| | | | | | 0.00) | |

TCR: Treatment to Control Ratio;
pTCR: non-parametric Tumor Control Ratio;
CI: Confidence Interval;
QD: every day;
Q7D: every seven day
TGI: Tumor growth inhibition

1-21. (canceled)

22. A method of treating multiple myeloma in a subject in need thereof, said method comprising administering to said subject a BET inhibitor and a proteasome inhibitor.

23. The method of claim 22, wherein the BET inhibitor is selected from the group consisting of 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146), INCB-054329, INCB-057643, GSK525762, GS-5829, CPI-0610, Birabresib, PLX51107, ABBV-075, BI 894999, FT-1101, ZEN-3694, GSK-2820151 and BMS-986158.

24. The method of claim 22, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146).

25. The method of claim 22, wherein the proteasome inhibitor is selected from the group consisting of bortezomib, carfilzomib, ixazomib, oprozomib, delanzomib and marizomib.

26. The method of claim 22, wherein the proteasome inhibitor is bortezomib.

27. The method of claim 22, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146), and the proteasome inhibitor is bortezomib.

28. The method of claim 22, wherein the method comprises administering a therapeutically effective amount of the BET inhibitor.

29. The method of claim 22, wherein the method comprises administering a therapeutically effective amount of the proteasome inhibitor.

30. The method of claim 22, wherein the method comprises administering a therapeutically effective amount of the BET inhibitor and a therapeutically effective amount of the proteasome inhibitor.

31. The method of claim 22, wherein the BET inhibitor and the proteasome inhibitor are each co-administered in separate dosage forms.

32. The method of claim 22, wherein the BET inhibitor is administered subcutaneously.

33. The method of claim 22, wherein the proteasome inhibitor is administered subcutaneously or intravenously.

34. The method of claim 22, further comprising administering one or more additional therapeutic agents selected from the group consisting of cytotoxic, chemotherapeutic and anti-cancer agents.

35. A pharmaceutical composition comprising a BET inhibitor and a proteasome inhibitor and one or more pharmaceutically acceptable excipients.

36. The pharmaceutical composition of claim 35, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146), and the proteasome inhibitor is bortezomib.

37. A method of treating multiple myeloma in a subject in need thereof, said method comprising administering to said subject the pharmaceutical composition of claim 35.

38. A kit comprising a BET inhibitor and a proteasome inhibitor for the simultaneous, separate or sequential administration of said BET inhibitor and proteasome inhibitor.

39. The kit of claim 38, wherein the BET inhibitor is selected from the group consisting of 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146), INCB-054329, INCB-057643, GSK525762, GS-5829, CPI-0610, Birabresib, PLX51107, ABBV-075, BI 894999, FT-1101, ZEN-3694, GSK-2820151 and BMS-986158.

40. The kit of claim 38, wherein the proteasome inhibitor is selected from the group consisting of bortezomib, carfilzomib, ixazomib, oprozomib, delanzomib and marizomib.

41. The kit of claim 38, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146), and the proteasome inhibitor is bortezomib.
